(19) United States
(12) Patent Application Publication
(10) Pub. No.: US 2023/0139888 A1
(43) Pub. Date: May 4, 2023

(54) MOISTURIZING PEEL CREAM FOR ENHANCING SKIN

(71) Applicant: L'OREAL, Paris (FR)

(72) Inventors: Zhengzheng LIAO, Cranford, NJ (US); Rebecca CHEN, Princeton, NJ (US); Zachary MARON, Jersey City, NJ (US)

(73) Assignee: L'OREAL, Paris (FR)

(21) Appl. No.: 17/976,360

(22) Filed: Oct. 28, 2022

Related U.S. Application Data
(60) Provisional application No. 63/273,538, filed on Oct. 29, 2021.

(30) Foreign Application Priority Data
Jan. 24, 2022 (FR) 22 00551

(51) Int. Cl. A61K 8/81; A61K 8/365; A61Q 19/08; A61Q 19/02

(52) U.S. Cl. CPC A61K 8/8164; A61K 8/365; A61Q 19/08; A61Q 19/02; A61K 2800/28; A61K 2800/34; A61K 2800/87

(57) ABSTRACT

A cosmetic skin-enhancing moisturizing peel cream composition includes at least one hydroxy acid, including alpha-hydroxy acid, present in an amount that is at least 5.5% by weight of the composition, at least one lightweight emollient selected from polar emollients and liquid alkanes, a blend of surfactants including one or all of cetearyl alcohol, ceteareth-20, glyceryl stearate and PEG-100 stearate, at least one thickening polymer present in an amount that is at least 3% by weight, and water and a water based solvent present from about 40% to about 75% by weight. The moisturizing peel cream composition is an oil in water emulsion provided in a creamy leave on form that is free or essentially free from silicones comprising a lamellar liquid crystal structure and has a pH of about 3.5 to about 4.1, provided for leave-on application to skin according to a daily treatment regimen of at least one day to up to twenty eight (28) days or more.

### REFERENCE TO RELATED APPLICATIONS

This application claims the benefit of, and priority to, U.S. Patent Application No. 63/273,538 filed on Oct. 29, 2021, and French Application No. FR2200551 filed on Jan. 24, 2022, the entireties of which are incorporated herein by reference.

### FIELD OF THE INVENTION

The present invention is generally directed to a cosmetic composition for enhancing skin appearance, including improving visual skin brightness and smoothness, minimizing pore appearance, and reducing the appearance fine lines and wrinkles, and a skin-enhancing regimen and article of manufacture for using the cosmetic skin-enhancing system. The moisturizing peel cream is especially suited for application to skin anywhere on the body, particularly on facial skin, in some particular embodiments, generally avoiding the skin under and around the eyes.

### BACKGROUND OF THE INVENTION

Chemical peels are a non-invasive dermatological products and approaches to improve and treat skin conditions including: photodamaged skin, hyperpigmentation, acne vulgaris, rosacea, premalignant skin cancer, wrinkles and fine lines, superficial scars and the like. These cosmetic compositions include a solution of acids that, when applied topically, penetrate into the skin, inducing and accelerating skin's natural regeneration process by sloughing off dead top layers of skin. The strength and efficacy of a peel is dictated by the formulation, acid type and concentration, which factors affect depth of penetration into the skin. Common chemical peels compositions include one or more of glycolic acid, lactic acid, salicylic acid, and trichloroacetic acid (TCA), among other ingredients. The efficacy of chemical peels is driven by the concentration of hydroxy acids (typically alpha- and beta-hydroxy acids) AHA/BHA and pH of the formula.

Professional-type peels include high levels of acid, for example, greater than 30%, and extreme low pH, typically less than 3, and are typically limited to use with licensed dermatologists and/or aestheticians. The key benefit of professional-type peels is that they confer relatively rapid and significant rejuvenation effects that can include one or more of improved skin radiance/brightness and pore appearance, skin smoothness and reduced fine lines and wrinkles. But professional-type peels can be expensive, are not as convenient as in-home use, and cause relatively high irritation and possible “down time” between applications. At-home-type peels that are used in the home market typically include alpha hydroxy acid (glycolic, lactic acid, mandelic acid, etc.) at a concentration of less than 10%, with a final pH that is greater than 3.0. The key benefits of at-home type peels include consumer convenience and possible cost savings; however, the rejuvenation results normally take around a month to achieve and are typically less impactful than professional-type peels.

Another challenge with chemical peels, in particular home chemical peels, is providing a hydrating, emollient composition with sufficiently high acids to accomplish meaningful benefits. It is known in the art that achieving a stable oil-in-water emulsion with high percentage of acids is challenging, at least in part as a result of thermodynamic instability associated with emulsion-acid interaction. Typically, this issue is addressed using relatively large amounts of electrolytes, surfactants and thickeners. These ingredients can yield a composition that is tacky, drying, and uncomfortable on the skin, and thus limits the viability for leave-on use. A common solution is to add organosilicon such as dimethicone, which acts as a sensory modifier. Due to some consumer preferences and perceptions relating to toxicity and environmental friendliness, more natural type alternatives to silicones are desirable.

Consumers seek skin products that provide a rich skin finish that is non-tacky and hydrating while effectively delivering the benefits of the acid actives. Thus, a need exists for a moisturizing peel cream that provides exceptional results such as those seen with professional peel compositions, with minimized or no adverse side effects and need for down time as compared with professional peel compositions, and with the sensorial and hydrating benefits of a rich emulsion. As disclosed herein, the inventors have developed a moisturizing peel cream composition and regimens of use address this need.

### BRIEF SUMMARY OF THE INVENTION

The summary is provided to introduce a selection of concepts in a simplified form that are further described below in the detailed description of the invention. This summary is not intended to identify key features of the claimed subject matter, nor is it intended to be used as an aid in determining the scope of the claimed subject matter.

In various embodiments, compositions are provided for use according to the invention that include a moisturizing peel cream provided for leave-on application to skin according to a daily treatment regimen of at least one day to up to twenty eight (28) days or more.

In some embodiments, the moisturizing peel cream is provided in a creamy oil in water emulsion a leave on form. In the various embodiments, water is present in the composition in a range from about 40% to about 75%. In some embodiments, the moisturizing peel cream has a pH of about 3.5 to about 4.1.

In various embodiments, the moisturizing peel cream includes at least one hydroxy acid, a blend of surfactants, at least one light-weight emollient, water at least one polymer thickener, and optionally one or more additional emollients.

In some embodiments, the hydroxy acid is selected from the group consisting of alpha-hydroxy acids, beta-hydroxy acids, poly-hydroxy acids, and combinations thereof. In some embodiments, the hydroxy acid in the moisturizing peel cream comprises at least one alpha-hydroxy acid present from at least about 5.5% to about 15% by weight, based upon the total weight of the moisturizing peel cream. In some embodiments, the hydroxy acid in the moisturizing peel cream comprises at least one alpha-hydroxy acid present from at least about 10% to about 15% by weight, based upon the total weight of the moisturizing peel cream. In some embodiments, the at least one alpha-hydroxy acid comprises glycolic acid.

In some embodiments, the hydroxy acid in the moisturizing peel cream further comprises at least one poly-hydroxy acid present from about 1% to about 15% by weight, based upon the total weight of the moisturizing peel cream. In some embodiments, the at least one poly-hydroxy acid comprises gluconolactone.

In some embodiments, the hydroxy acid in the moisturizing peel cream further comprises at least one beta-hydroxy acid present from about 0.1% to about 2% by weight, based upon the total weight of the moisturizing peel cream. In some embodiments, the at least one beta-hydroxy acid comprises salicylic acid.

In some embodiments, the moisturizing peel cream includes two or more hydroxy acids selected from the group consisting of alpha-hydroxy acids, poly-hydroxy acids, beta-hydroxy acids, and combinations thereof. In some embodiments, the hydroxy acid in the moisturizing peel cream further comprises at least one beta-hydroxy acid present from about 0.1% to about 2% by weight, or at least one poly-hydroxy acid present from about 1% to about 15% by weight, the amounts based upon the total weight of the moisturizing peel cream. In some embodiments, the moisturizing peel cream includes an alpha-hydroxy acid comprising glycolic acid and a poly-hydroxy acid comprising gluconolactone.

In some embodiments, the blend of surfactants is present in a range from about 4.5% to about 8%. In some embodiments, the blend of surfactants includes one or a combination of cetearyl alcohol and ceteareth-20, glyceryl stearate and PEG-100 stearate.

In some embodiments, the at least one light-weight emollient is present in a range from about 1% to about 12%. In some embodiments the at least one light-weight emollient comprises coco-caprylate/caprate.

In some particular embodiments, the moisturizing peel cream includes coco-caprylate/caprate, or C15-19 alkane or a combination of these and the blend of surfactants that comprises cetearyl alcohol and ceteareth-20, glyceryl stearate and PEG-100 stearate

And the emulsion contains about 3% of at least one or more polymer as thickening reagents. In some embodiments, the polymers may include aluminum starch octenylsuccinate a shine control agent and sensory modifier, one or more emulsifying thickener, such as Hydroxyethyl Acrylate/Sodium Acryloyldimethyl Taurate Copolymer.

In some embodiments, the moisturizing peel cream optionally includes from about 5% to about 20% of one or a combination of other emollients. In some such embodiments that include other emollients, the emollients may be selected from plant butters such as cocoa butter, rhea butter, and caprylic/capric triglyceride, C12-15 alkyl benzoate, sunflower seed oil, and combinations of these.

In some embodiments, the composition may comprise one or more additional ingredients selected from the group consisting of chelating agents, pH adjusters, humectants, antioxidants, plant extracts, plant oils and butters, active components, essential oils, vitamins, antimicrobials and preservatives, fragrances, pearlescent agents, odor absorbers, coloring materials, fillers such as clays, talc, powders, including spherical/silica powders and mineral powders, carbohydrate based gellifying agents, organic thickeners, penetrants, sequestrants, fragrances, dispersants, film-forming agents, ceramides, opacifiers, and combinations thereof.

The moisturizing peel cream emulsion is free or essentially free from organosilicon such as dimethicone and cyclopentasiloxane.

In various embodiments, the moisturizing peel cream composition is contained to be dispensed for application to skin one time, or according to a regimen of daily application. In some embodiments, the moisturizing peel cream composition is provided in an article of manufacture comprising a container for dispensing the composition, wherein the container is a multi-use or a single use container. In some embodiments, the article of manufacture comprises aliquots of the moisturizing peel cream composition, each separately contained as single units in a plurality of single use containers.

The invention also provides, in various embodiments, a regimen for enhancing skin comprising applying the moisturizing peel cream to skin, including anywhere on the body or on the face. In some embodiments, application on the face may be limited to a region of skin excluding areas of skin around an eye.

In some embodiments, the moisturizing peel cream may be used according to a regimen that includes periodic application of a composition that contains peroxide or another oxidizing agent (an “oxidizing component”). Application of the moisturizing peel cream and oxidizing component may be according to any one of a number of possible application cycles wherein the moisturizing peel cream is used in conjunction with the oxidizing component, applied together, or applied in sequence, and at a frequency of once or, or more frequently, with applications of at least one of the compositions on a daily basis for a few or several days.

In some embodiments, the steps of the regimen further include rinsing the skin after application of at least one of the oxidizing component and the moisturizing peel cream, and include the steps in any order, and wherein the step of rinsing may be repeated.

In some embodiments, the moisturizing peel cream and the oxidizing component are applied sequentially, in any order. In some embodiments, the oxidizing component is applied first, followed by the moisturizing peel cream, and in other embodiments, the moisturizing peel cream is applied first, followed by oxidizing component. In some embodiments, the moisturizing peel cream and the oxidizing component are applied together. In some embodiments, a regimen according to the disclosure may include first application of the oxidizing component on a first day, optionally including application of the moisturizing peel cream on the first day, and thereafter followed by daily applications of the moisturizing peel cream for two or more days.

In some embodiments, the step of applying the oxidizing component is repeated once every third, fourth or fifth day and wherein the step of applying the moisturizing peel cream is skipped on the days that the step of applying the first composition is repeated.

In some embodiments, the regimen is practiced for at least 28 days. The regimen may be selected from one or a combination of application cycles.

Other features and advantages of the present invention will be apparent from the following more detailed description, by way of example, the principles of the invention.

This disclosure describes exemplary embodiments in accordance with the general inventive concepts and is not intended to limit the scope of the invention in any way. Indeed, the invention as described in the specification is broader than and unlimited by the exemplary embodiments set forth herein, and the terms used herein have their full ordinary meaning.

### BRIEF DESCRIPTION OF THE DRAWINGS

This application contains at least one drawing executed in color. Copies of this patent or patent application publication with color drawing(s) will be provided by the Office upon request and payment of the necessary fee.

Features and advantages of the general inventive concepts will become apparent from the following description made with reference to the accompanying drawings, including drawings represented herein in the attached set of figures, of which the following is a brief description:

FIG. 1 includes color photographic microscopy images of the oil in water emulsion form of the moisturizing peel cream demonstrating the lamellar liquid crystal structure in an embodiment that includes starch thickener; and

FIG. 2 includes a color photographic microscopy image of a control composition demonstrating lack of lamellar liquid crystal structure.

This disclosure describes exemplary embodiments in accordance with the general inventive concepts and is not intended to limit the scope of the invention in any way. Indeed, the invention as described in the specification is broader than and unlimited by the exemplary embodiments set forth herein, and the terms used herein have their full ordinary meaning.

In accordance with the various embodiments, any aspect of any embodiment can be combined or used with any other unless explicitly stated to be exclusive or exclusionary.

### DETAILED DESCRIPTION OF THE INVENTION

As used herein, the term “enhancing” of skin refers to effects on skin as a result of use of the inventive moisturizing peel cream that can include one or more of improved or reduced pore appearance, improved visual skin smoothness, improved skin brightness, and reduced fine lines and wrinkles.

The term “silicone-free” as used herein means containing less than 5%, such as less than 3%, such as less than 1% by weight, based on the total active weight of the composition, of silicones, such as silicone polymers, for example selected from dimethicone, cyclopentasiloxane, cyclohexasiloxane and other silicone oils, and silicone elastomers.

As described and exemplified in various embodiments herein, the inventors have created a moisturizing peel cream in the form of an oil-in-water emulsion with at least 5.5% or more of hydroxy acids that has a rich creamy texture without the use of silicone. The hydroxy acids are selected from the group consisting of alpha-hydroxy acids and polyhydroxy acids or combinations thereof. In some embodiments, the moisturizing peel cream has a pH of about 3.5 to about 4.1. The emulsion further contains from about 4.5% to about 8% of a blend of surfactants. In addition, the emulsion further contains from about 1% to about 12% of at least one light-weight emollient, in some embodiments comprising coco-caprylate/caprate, and optionally from about 5% to about 20% of one or a combination of other emollients, and from about 40% to about 75% water. In some embodiments, the blend of surfactants includes one or a combination of cetearyl alcohol and ceteareth-20, glyceryl stearate and PEG-100 stearate. In some particular embodiments, the moisturizing peel cream includes coco-caprylate/caprate, or C15-19 alkane or a combination of these and the blend of surfactants that comprises cetearyl alcohol and ceteareth-20, glyceryl stearate and PEG-100 stearate. In some such embodiments that include other emollients, the emollients may be selected from plant butters such as cocoa butter and rhea butter, caprylic/capric triglyceride, C12-15 alkyl benzoate, sunflower seed oil, and combinations of these. And the emulsion contains about 3% of at least one or more polymer as thickening reagents. In some embodiments, the polymers may include aluminum starch octenylsuccinate a shine control agent and sensory modifier, one or more emulsifying thickener, such as Hydroxyethyl Acrylate/Sodium Acryloyldimethyl Taurate Copolymer.

The moisturizing peel cream emulsion is free or essentially free from organosilicon such as dimethicone and cyclopentasiloxane.

As shown in the examples, the inventors have provided a moisturizing peel cream that confers the benefits of one or more of improved or reduced pore appearance, improved visual skin smoothness, improved skin brightness, and reduced fine lines and wrinkles. The observed benefits are realized when used over a period from about 14 days up to 28 days or more and are comparable to the benefits seen with high acid professional type peels, without the inconvenience and irritation. The moisturizing peel cream is especially suited for application to skin anywhere on the body, particularly on facial skin, and in some embodiments on facial skin generally avoiding the skin under and around the eyes.

As exemplified herein, the inventors have shown in stratum corneum (SC) studies that use of the moisturizing peel cream provides immediate exfoliation and brightening after one application. In addition, in a clinical study the inventors have shown improvement in skin brightness, visual skin smoothness, and reduced pigmentation are observed after 14 days of use of the moisturizing peel cream, and improvement in pore appearance after 28 days of use. The study clinical study involved an application cycle of daily use of the moisturizing peel cream.

These unexpected benefits of skin brightness, visual smoothness, reduced pore size are attained according to the instant invention in a time period than is comparable with conventional home chemical peels, with less irritation than is observed with professional chemical peels.

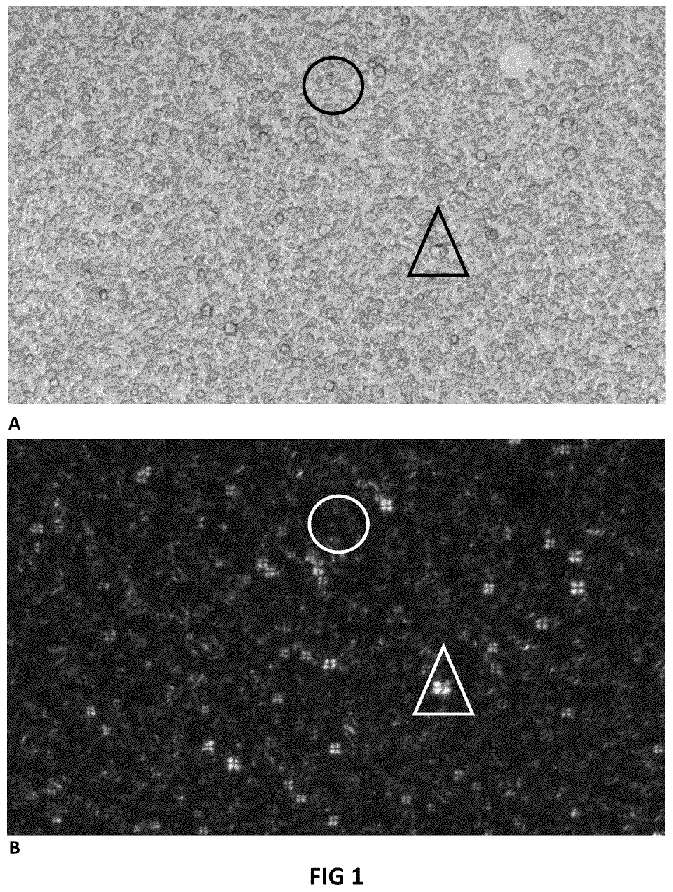

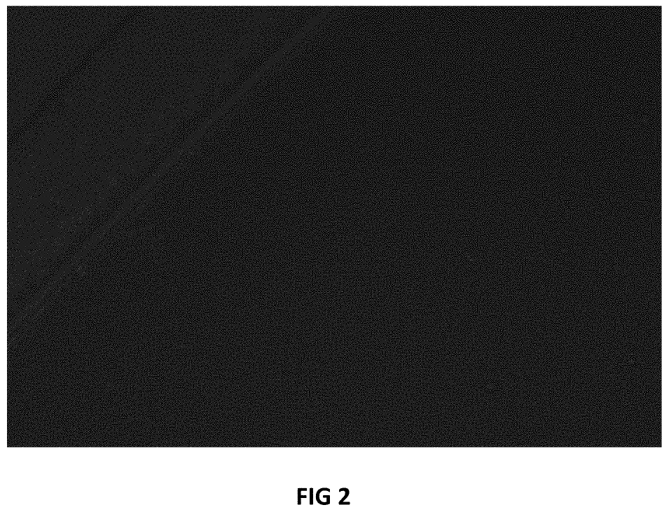

In some embodiments, the moisturizing peel cream includes an emulsion that includes a lamellar structure, for example a lamellar liquid crystal structure, that includes the at least one lightweight emollient, for example a polar oil or a liquid alkane, in the blend of surfactants. Demonstration of the presence or absence of lamellar structure included maintaining examined compositions at 45° C. for two weeks, followed by imaging with dark field microscopy at room temperature. Referencing now to the drawings, The inventive composition, INV 1, shown in Table 2 and corresponding to the dark field microscopy image of FIG. 1, shows evidence of lamellar structure, bright and dark field microscopy images show large lamellar liquid crystals (shown in circles) formed around oil droplets (6% coco-caprylate/caprate) wherein starch powder particles (shown in triangles) are dispersed throughout. The test composition as disclosed in Example 2 herein below, shown in Table 5 and corresponding to the dark field microscopy image of FIG. 2, shows a similar image in a composition that lacks the lamellar liquid crystal structure.

When applied to skin, during rub-in, the lamellar structure around the oil droplet in contact with skin ensures good spreadability and rich skin feel. When the oil droplet is spread on skin and continued friction is applied by finger, the emollient delivers a silky and slippery skin feel that is desirable to consumer and eliminates the need to use organosilicon for creating this type of sensory benefit.

The moisturizing peel cream also includes one or more additional ingredients selected from the group consisting of chelating agents, pH adjusters, humectants, antioxidants, plant extracts, plant oils and butters, active components, essential oils, vitamins, antimicrobials and preservatives, fragrances, pearlescent agents, odor absorbers, coloring materials, fillers such as clays, talc, powders, including spherical/silica powders and mineral powders, carbohydrate based gellifying agents, organic thickeners, penetrants, sequestrants, fragrances, dispersants, film-forming agents, ceramides, opacifiers, and combinations thereof.

Without being bound by theory, the inventors posit that the moisturizing peel cream exhibits stability of the oil in water emulsion even in the presence of high acid levels (>=10% total acids, including alpha- and poly-hydroxy acids) by forming a lamellar gel network around oil droplets using a blend of surfactants that includes a combination of cetearyl alcohol, ceteareth-20, glyceryl stearate, and PEG-100 stearate. It is further posited that the lamellar gel network gives the emulsion high viscosity and acts as a physical barrier to prevent the coalescence of oil droplets, thus enhancing the stability of emulsion. It is further posited that participation of acids in the lamellar structure, the acids are slowly released to skin after applied, which results in achieving lower irritation. And it is further posited that the lamellar gel network can retain water molecules better than a standard emulsion, thus, when applied on skin, it can achieve better skin hydration, which also contributes to reduced irritation as compared with other peels.

In some particular embodiments, the moisturizing peel cream is a creamy emulsion form that may be thick or liquidous (flowable). In some particular embodiments, the moisturizing peel cream is a liquidous toner form. The moisturizing peel cream can be provided in a creamy emulsion form, for example as a leave-on form or a rinse-off mask. Other formulations are possible, and the provided examples are non-limiting.

In accordance with the various embodiments, it is contemplated that some or all of following ingredients would be included in the compositions used according to the invention:

Moisturizing Peel Cream of the Skin-Enhancing Treatment

Accordingly, the various ingredients that may be present in the moisturizing peel cream are provided herein below or further herein in the section described as other ingredients, and as shown in the Examples herein.

Alpha Hydroxy Acid

In accordance with the various embodiments, the moisturizing peel cream comprises at least one alpha hydroxy acid.

Suitable alpha hydroxy acids include glycolic acid, lactic acid, tartaric acid, mandelic acid, citric acid, ester derivatives thereof and combinations thereof. Exemplary ester derivatives include ester compounds of glycolic acid, lactic acid, such as methyl lactate, ethyl lactate, butyl lactate and, similarly, ester compounds of glycolic acid, tartaric acid, mandelic acid, citric acid. One particularly suitable alpha hydroxy acid is lactic acid. In some embodiments, lactic acid, or 2-hydroxypropanoic acid, is provided to the chemical peel composition to provide enhanced exfoliation of the skin. In addition, lactic acid also boosts production of glycosaminoglycan (GAG) in the skin, improving the barrier function and moisturization of skin.

In some particular embodiments, the alpha hydroxy acid in the moisturizing peel cream comprises glycolic acid.

The moisturizing peel cream includes a concentration of alpha hydroxy acid in a range from about at least about 5%, or from about 5.5% to about 15%, or from about 5.5% to about 14% or from about 5.5% to about 10%, or from about 10% to about 12%, or is about 5.5%, or is about 10%, or any suitable combination, sub-combination, range, or sub-range thereof by weight, based on the weight of the moisturizing peel cream. One of ordinary skill in the art, however, will appreciate that other ranges are within the scope of the invention. In accordance with some embodiments, the amount of alpha hydroxy acid present is not more than about 10%.

Thus, any one of or a combination of alpha hydroxy acid is present, by weight, based on the total weight of the moisturizing peel cream, from about 5, 5.5, 6,6.5, 7, 7.5, 8, 8.5, 9, 9.5, 10, 10.5, 11, 11.5, 12, 12.5, 13, 13.5, 14, 14.5 to about 15 weight percent, including increments and ranges therein and there between.

Poly Hydroxy Acid

In accordance with the various embodiments, the moisturizing peel cream may comprise at least one poly hydroxy acid.

Suitable poly hydroxy acids include gluconolactone and derivatives thereof and combinations thereof.

The moisturizing peel cream may include a concentration of poly hydroxy acid in a range from about 4% to about 15%, or from about 8% to about 14% or from about 9% to about 13%, or from about 10% to about 12%, or is about 10%, or any suitable combination, sub-combination, range, or sub-range thereof by weight, based on the weight of the moisturizing peel cream. One of ordinary skill in the art, however, will appreciate that other ranges are within the scope of the invention. In accordance with some embodiments, the amount of poly hydroxy acid present is not more than about 10%.

Thus, any one of or a combination of poly hydroxy acid, when present, is present, by weight, based on the total weight of the moisturizing peel cream, from about 4, 5, 6, 7, 8, 9, 10, 11, 12, 13, 14, to about 15 weight percent, including increments and ranges therein and there between.

Beta Hydroxy Acid

In accordance with the various embodiments, the moisturizing peel cream may comprise at least one beta hydroxy acid. In some particular embodiments, the moisturizing peel cream may include a beta hydroxy acid comprising salicylic acid.

The term “beta-hydroxy acid” is understood to mean, according to the present invention, a carboxylic acid having a hydroxyl functional group and a carboxylic functional group separated by two carbon atoms. A beta hydroxy acid can be present in the moisturizing peel cream in the form of the free acid and/or in the form of one of its associated salts (salts with an organic base or an alkali metal, in particular), especially according to the final pH imposed on the moisturizing peel cream.

Suitable beta hydroxy acids include salicylic acid and derivatives thereof (including 5-n-octanoylsalicylic acid, salicylate, sodium salicylate, and willow extract), capryloyl salicylic acid, beta hydroxybutanoic acid, propionic acid, beta-hydroxy beata-methylbutyric acid, carnitine tropic acid, and trethocanic acid, and combinations of these.

And in some particular embodiments, the beta hydroxy acid in the moisturizing peel cream excludes at least one of salicylic acid and derivatives thereof (including 5-n-octanoylsalicylic acid, salicylate, sodium salicylate, and willow extract), capryloyl salicylic acid, beta hydroxybutanoic acid, propionic acid, beta-hydroxy beata-methylbutyric acid, carnitine tropic acid, and trethocanic acid.

The moisturizing peel cream may include a concentration of beta hydroxy acid in a range from about 0.1% up to and not more than about 2% of beta hydroxy acid, by weight, based on the total weight of the moisturizing peel cream. In some embodiments, the moisturizing peel cream includes up to and not more than about 2%, or about 1.9% of beta hydroxy acid. In some embodiments, the moisturizing peel cream includes up to and not more than about 1% of beta hydroxy acid. In accordance with some embodiments, the amount of beta hydroxy acid present is not more than about 0.40% to about 0.50%. In some embodiments, the moisturizing peel cream includes from about 0.1% to about 1% of beta hydroxy acid, or from about 0.2% to about 2.0%, or from about 0.1% to about 1.5%, or from about 0.2% to about 1.5%, or from about 0.3% to about 1.0%, or from about 0.35% to about 0.75%, or from about 0.4% to about 0.5%, or is about 0.45%, or any suitable combination, sub-combination, range, or sub-range thereof by weight, based on the weight of the moisturizing peel cream. One of ordinary skill in the art, however, will appreciate that other ranges are within the scope of the invention.

Thus, the at least one beta hydroxy acid, when present, is present in the moisturizing peel cream from about 0.1, 0.2, 0.3, 0.4, 0.5, 0.6, 0.7, 0.8, 0.9, 1.0, 1.2, 1.3, 1.4, 1.5, 1.6, 1.7, 1.8, 1.9, to about 2.0 weight percent, including increments and all ranges and subranges therein and there between.

Phenylethyl Resorcinol

In accordance with some embodiments, the moisturizing peel cream may comprise phenylethyl resorcinol.

Phenylethyl resorcinol functions a tyrosinase inhibitor. The phenylethyl resorcinol, when utilized in the moisturizing peel cream effectively whiten skin and reduce skin tone unevenness. Phenylethyl resorcinol has not shown adverse effects in basic toxicological tests, including acute oral toxicity, mutagenicity, skin irritation, skin sensitization, and phototoxicity.

In accordance with the various embodiments, when present, the amount of phenylethyl resorcinol present in the moisturizing peel cream is from about 0.2% to about 2.0%, or from about 0.2% to about 1.5%, or from about 0.4% to about 1.0%, or from about 0.6% to about 0.9%, or from about 0.7% to about 0.8%, or from about 0.2% to about 0.8%, is about 0.75%, or any suitable combination, sub-combination, range, or sub-range thereof by weight, based on the weight of the moisturizing peel cream. One of ordinary skill in the art, however, will appreciate that other ranges are within the scope of the invention. In accordance with some embodiments, the amount of phenylethyl resorcinol present is not more than about 0.2% to about 0.8%.

Thus, phenylethyl resorcinol, when present in the moisturizing peel cream, is present, by weight, based on the total weight of the moisturizing peel cream, from about 0.20, 0.30, 0.40, 0.50, 0.60, 0.70, 0.80, 0.90, 1.0, 1.1, 1.2, 1.3, 1.4, 1.5, 1.6, 1.7, 1.8, 1.9 to about 2.0 weight percent, including increments and ranges therein and there between.

Oil/Emollient

In accordance with the various embodiments, the moisturizing peel cream includes one or more oils.

In the various embodiments, the moisturizing peel cream includes at least one lightweight emollient selected from polar emollients and linear liquid alkanes. In some particular embodiments, the composition comprises one or a combination of coco-caprylate/caprate or C15-19 alkane.

The at least one lightweight emollient is present in a range from about 0.5% to about 15%, or from about 1% to about 12%, or from about 1% to about 10%, or from about 2% to about 8%, or any suitable combination, sub-combination, range, or sub-range thereof by weight, based on the weight of the moisturizing peel cream. One of ordinary skill in the art, however, will appreciate that other ranges are within the scope of the invention.

Other suitable polar emollients may be selected from the group consisting of Isopropyl myristate, dicaprylyl ether, ethylhexyl palmitate, Isopropyl palmitate, cetearyl ethylhexanoate, lsononyl isononanoate, isopropyl isostearate, diisopropyl sebacate, coco caprylate/caprate, diisopropyl adipate, and combinations thereof.

Other suitable linear liquid alkanes (C11 to C20) may be selected from the group consisting of isohexadecane, C15-19 alkane, isododecane, undecane, tridecane, dodecane, and combinations thereof.

In some particular embodiments, the chemical peel component includes coco-caprylate/caprate, C15-19 alkane, dodecane and combinations thereof.

In some particular embodiments, the moisturizing peel cream includes coco-caprylate/caprate, C15-19 alkane and dodecane.

Thus, any one of or a combination of lightweight emollient is present, by weight, based on the total weight of the moisturizing peel cream, from about 0.5, 0.6, 0.7, 0.8, 0.9, 1, 2, 3, 4, 5, 6 , 7, 8, 9, 10, 11, 12, 13, 14, to about 15 weight percent, including increments and ranges therein and there between.

In some embodiments, the moisturizing peel cream may further include at least one oil selected from the group consisting of Helianthus annuus (sunflower) seed oil, Sesamum indicum (sesame) seed oil, coconut oil, jojoba oil, isopropyl palmitate, isononyl isononanoate, apricot oil, dicaprylyl capric triglyceride, dicaprylyl carbonate, C12-15 alkyl benzoate, caprylic/capric triglyceride and combinations thereof.

More generally, in the various embodiments, the composition may include additional oil, which, when present, is generally immiscible in water, and may be selected from hydrocarbons, silicones, fatty alcohols, glycols and vegetable oils. The oil may include one or a combination of polar and non-polar oil. In some embodiments, the oil may be chosen from hydrocarbon-based oils from plants or of plant origin, mineral oil, ester oils, fatty alcohols containing from 12 to 26 carbon atoms, fatty acids containing from 12 to 26 carbon atoms and vinylpyrrolidone copolymers, and combinations thereof. In some particular embodiments, an oil may be chosen from purcellin oil (cetostearyl octanoate), squalane, hemisqualane, isononyl isononanoate, C12 to C15 alkyl benzoate, 2-ethylhexyl palmitate, isodecyl neopentanoate, tridecyl neopentanoate, octyldodecyl neopentanoate, 2-octyldodecyl stearate, 2-octyldodecyl erucate, ( )eyl erucate, isostearyl isostearate, 2-octyldodecyl benzoate, alcohol or polyalcohol octanoates, decanoates or ricinoleates, isopropyl myristate, isopropyl palmitate, butyl stearate, hexyl laurate, 2-ethylhexyl palmitate, 2-hexyldecyl laurate, 2-octyldecyl palmitate, 2-octyldodecyl myristate and 2-diethylhexyl succinate, cocoglyceride, cyclomethicone, dimethicone, dicaprylyl carbonate, dicapryl maleate, caprylic/capric triglyceride, isopropyl myristate, octyl stearate, isostearyl linoleate, lanolin oil, coconut oil, cocoa butter, olive oil, avocado oil, aloe extracts, jojoba oil, castor oil, fatty acid, oleic acid, stearic acid, fatty alcohol, cetyl alcohol, hexadecyl alcohol, diisopropyl adipate, hydroxybenzoate esters, benzoic acid esters of C9-C15 alcohols, alkanes, mineral oil, silicone, dimethyl polysiloxane, ether, polyoxypropylene butyl ether, polyoxypropylene cetyl ether, C12-C15 alkyl benzoate, aryl alkyl benzoate, Isopropyl Lauroyl sarcosinate, and any combinations thereof.

In accordance with the various embodiments, additional oil, when present, in the moisturizing peel cream is at present from about 1% to about 20%. One of ordinary skill in the art, however, will appreciate that other ranges are within the scope of the invention. The moisturizing peel cream may include more than one oil, such as a mixture of oils wherein one or the mixture of oils is present in an amount that alone or combined is present from about 1% to about 20% by weight based on the weight of the moisturizing peel cream, and wherein the total amount of additional oil is present up to about 20% of the moisturizing peel cream.

In accordance with some embodiments, the moisturizing peel cream may comprise at least one plant butter, the at least one butter selected from the group consisting of Butyrospermum parkii (rhea) butter, Theobroma cacao (cocoa) seed butter, almond butter, aloe butter, apricot kernel butter, avocado butter, coconut cream butter, cupuacu butter, dhupu butter, hemp butter, jojoba butter, kokum butter, macadamia nut butter, mango butter and combinations thereof.

In accordance with the various embodiments, the amount of butter present in the composition may be at least 1%, alternatively from about 1% to about 10%; alternatively, from about 2% to about 9%, alternatively from about 3% to about 8%, alternatively from about 4% to about 7%, alternatively from about 5% to about 6%, or any suitable combination, sub-combination, range, or sub-range thereof by weight, based on the weight of the moisturizing peel cream. One of ordinary skill in the art, however, will appreciate that other ranges are within the scope of the invention.

In some embodiments, the at least one butter has a melting point in the range of from about 30 to about 45 degrees Celsius.

Thus, any one of or a combination of oils may be present, by weight, based on the total weight of the moisturizing peel cream, from about 1, 2, 3, 4, 5, 6 , 7, 8, 9, 10, 11, 12, 13,14, 15, 16, 17, 18, 19, to about 20 weight percent, including increments and ranges therein and there between.

Surfactant

In accordance with the various embodiments, the moisturizing peel cream includes a blend of surfactants comprising one or a combination of surfactants, for example selected from the group consisting of stearic acid, cetearyl alcohol, palmitic acid, myristic acid, polysorbate 20, glyceryl stearate (and) peg-100 stearate, cetearyl alcohol (and) ceteareth-20, and glyceryl stearate, sorbitan stearate, and combinations thereof.

In some particular embodiments, the moisturizing peel cream includes a blend of surfactants comprising at least one or more of stearic acid, cetearyl alcohol, palmitic acid, myristic acid, polysorbate 20, glyceryl stearate (and) peg-100 stearate, cetearyl alcohol (and) ceteareth-20, or glyceryl stearate.

In accordance with the various embodiments, the amount of surfactant present in the composition may be from about 1% to about 20%, alternatively from about 2% to about 19%, alternatively from about 3% to about 18%, alternatively from about 4% to about 17%, alternatively from 4.5% to about 18%, alternatively from about 5% to about 16%, alternatively from about 6% to about 15%, alternatively from about 7% to about 14%, alternatively from about 8% to about 13%, alternatively from about 9% to about 12%, alternatively from about 10% to about 11%, or any suitable combination, sub-combination, range, or sub-range thereof by weight, based on the weight of the composition. One of ordinary skill in the art, however, will appreciate that other ranges are within the scope of the invention.

In some embodiments, surfactants selected are sulfate free. In some particular embodiments, the blend of surfactants comprises two or more surfactants that are sulfate-free. The term “sulfate-free” as used herein means that sulfate has not been added as a component. In some embodiments, a composition is devoid of sulfate. Those of skill in the art will appreciate that sulfate may be present in a composition via its presence in one or more of the formulation components; thus, in some embodiments a composition may be “essentially sulfate-free” wherein sulfate is present at a concentration that does not exceed 5% by weight, and in some instances is present not more than 3% by weight, and in some instances is present not more than 1% by weight, based on the weight of the cleansing composition. Thus, the term “sulfate-free” means that the surfactant does not include sulfate.

In accordance with the various embodiments, each one of the surfactants in the blend of surfactants is present in the composition in an amount from about 0.5% to about 20%, or from about 0.5% to about 8%, or from about 1% to about 4%, or any suitable combination, sub-combination, range, or sub-range thereof by weight, based on the weight of the moisturizing peel cream. In some embodiments, a blend of surfactants is present in the composition in an amount from about 5% to about 8%, or any suitable combination, sub-combination, range, or sub-range thereof by weight, based on the weight of the moisturizing peel cream. One of ordinary skill in the art, however, will appreciate that other ranges are within the scope of the invention.

Thus, each one of the surfactants, alone or in combination, is present in an amount by weight, based on the total weight of the moisturizing peel cream, from about 0.5, 0.6, 0.7, 0.8, 0.9, 1, 2, 3, 4, 5, 6, 7, 8, 9, 10, 11, 12, 13,14, 15, 16, 17, 18, 19, to about 20 weight percent, including increments and ranges therein and there between.

Solvent

In accordance with the various embodiments, water is present in the moisturizing peel cream in a range from about 40% to about 75%, or from about 45% to about 70%, or from about 48% to about 65%, or any suitable combination, sub-combination, range, or sub-range thereof by weight, based on the weight of the moisturizing peel cream. One of ordinary skill in the art, however, will appreciate that other ranges are within the scope of the invention.

Thus, water is present, by weight, based on the total weight of the moisturizing peel cream, from about 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74 to about 75 weight percent, including increments and ranges therein and there between.

The water used may be sterile demineralized water and/or a floral water such as rose water, cornflower water, chamomile water or lime water, and/or a natural thermal or mineral water such as, for example: water from Vittel, water from the Vichy basin, water from Uriage, water from La Roche Posay, water from La Bourboule, water from Enghien-les-Bains, water from Saint Gervais-les-Bains, water from Neris-les-Bains, water from Allevar-les-Bains, water from Digne, water from Maizieres, water from Neyrac-les-Bains, water from Lons-le-Saunier, water from Eaux Bonnes, water from Rochefort, water from Saint Christau, water from Les Fumades, water from Tercis-les-Bains or water from Avene. The water phase may also comprise reconstituted thermal water, that is to say a water comprising trace elements such as zinc, copper, magnesium, etc., reconstituting the characteristics of a thermal water.

The pH of the moisturizing peel cream is not limited but is generally not less than 3, and more particularly in a range from about 3 to about 4.5, or from about 3.5 to about 4.1, or about 3.8. The pH can be adjusted to the desired value by addition of a base (organic or inorganic) to the moisturizing peel cream, for example ammonia or a primary, secondary or tertiary (poly)amine, such as monoethanolamine, diethanolamine, triethanolamine, isopropanolamine or 1,3-propanediamine, or alternatively by addition of an inorganic or organic acid, advantageously a carboxylic acid, such as, for example, citric acid.

In some embodiments, the moisturizing peel cream includes one or more water based solvents selected from the group consisting of glycols, for example, glycerin and pentylene glycol, alcohols for solubilizing acid, for example, monoalcohols such as monohydric C₁-C₈ alcohols such as ethanol, propanol, butanol, isopropanol, isobutanol, and benzyl alcohol, and phenylethyl alcohol, and combinations thereof. In some embodiments, the water based solvents include glycerin and pentylene glycol. In some embodiments, glycerin is present in a range from about 5% to about 20%.

In accordance with the various embodiments, the amount of water based solvent present in the moisturizing peel cream is in the range from about 1% to about 20%, or from about 2% to about 18%, or from about 10% to about 15%, or any suitable combination, sub-combination, range, or sub-range thereof by weight, based on the weight of the moisturizing peel cream. One of ordinary skill in the art, however, will appreciate that other ranges are within the scope of the invention.

Thus, any one of ora combination of water based solvents may be present, by weight, based on the total weight of the moisturizing peel cream, from about 1, 2, 3, 4, 5, 6, 7, 8, 9, 10, 11, 12, 13, 14, 15, 16, 17, 18, 19, to about 20 weight percent, including increments and ranges therein and there between.

Thickener

In accordance with the disclosure, the moisturizing peel cream can include at least one thickener. In some embodiments the moisturizing peel cream comprises two or more thickeners. In some embodiments, the moisturizing peel cream includes one or a combination of thickeners selected from the group consisting of aluminum starch octenylsuccinate, xanthan gum, hydroxyethyl acrylate/sodium acryloyldimethyl taurate copolymer, and combinations thereof. In some particular embodiments, the moisturizing peel cream includes aluminum starch octenylsuccinate, xanthan gum, and hydroxyethyl acrylate/sodium acryloyldimethyl taurate copolymer.

More generally, in some embodiments, the moisturizing peel cream incudes one or more thickeners for a water-based system, selected from, for example, aluminum starch octenylsuccinate, xanthan gum, hydroxyethyl acrylate/sodium acryloyldimethyl taurate copolymer, acrylates/beheneth-25 methacrylate copolymer, acrylates copolymer, acrylates/C10-30 alkyl acrylate crosspolymer, sodium acrylates copolymer (and) caprylic/capric triglyceride, carbomer, hydroxypropyl guar, carrageenan, ceratonia siliqua (carob) gum, ammonium polyacryloyldimethyl taurate, ammonium acryloyldimethyltaurate/steareth-25 methacrylate crosspolymer, polyacrylate crosspolymer-6 and combinations thereof.

In accordance with those embodiments that include one or more thickeners, each thickener is present in the composition in an amount from about 0.01% to about 15%, or from about 1% to about 10%, or from about 2% to about 5%, or from about 1% to about 3%, or any suitable combination, sub-combination, range, or sub-range thereof by weight, based on the weight of the moisturizing peel cream. And in some embodiments, the total amount of thickener present in the composition is at least about 3%, or about 4%, or about 10% or more, or in an amount from about 3% to about 15%, from about 3% to about 10%, or from about 3% to about 5%, or any suitable combination, sub-combination, range, or sub-range thereof by weight, based on the weight of the moisturizing peel cream. One of ordinary skill in the art, however, will appreciate that other ranges are within the scope of the invention.

Thus, one or more thickener in the moisturizing peel cream, when present, is present by weight, based on the total weight of the moisturizing peel cream, from about 0.01, 0.02, 0.03, 0.04, 0.05, 0.06, 0.07, 0.08, 0.09, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 5, 6, 7, 8, 9, 10, 11, 12, 13, 14, to about 15 percent, including increments and ranges therein and there between.

Other Ingredients

In accordance with the various embodiments, the moisturizing peel cream may further include additional ingredients, generally including, but not limited to, chelating agents, pH adjusters, humectants, antioxidants, plant extracts, plant oils and butters, active components, essential oils, vitamins, antimicrobials and preservatives, fragrances, pearlescent agents, odor absorbers, coloring materials, fillers such as clays, talc, powders, including spherical/silica powders and mineral powders, carbohydrate based gellifying agents, organic thickeners, penetrants, sequestrants, fragrances, dispersants, film-forming agents, ceramides, opacifiers, and combinations thereof. Non limiting examples of some of these optional additional ingredients are provided herein below.

Chelating Agent

In some embodiments, one or more other components, such as chelating agents can be present in the moisturizing peel cream, in amounts from about 0.01% to about 2% by weight, from about 0.02% to about 1.5% by weight, from about 0.02% to about 1%, from about 0.02% to about 0.5%, and from about 0.025 to about 0.15%, or any suitable combination, sub-combination, range, or sub-range thereof by weight, based on the weight of the moisturizing peel cream or the moisturizing peel cream. In some exemplary embodiments, chelating agents are selected from ethylenediaminetetraacetic acid (EDTA), tetrasodium glutamate diacetate, tetrasodium etidronate, tetrasodium pyrophosphate, pentasodium ethylenediamine tetramethylene phosphonate, sodium staminate and combinations of these.

Thus, one or a combination of chelating agents may be present, by weight, based on the total weight , each one or the combination present from about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0 up to about 2 weight percent, including increments and ranges therein and there between.

Optional Actives and Other Ingredients

In some embodiments, there may be one or more additives present in the moisturizing peel cream the additive selected from, for example, humectants, such as acetamide MEA, glycols, such as glycerin and propylene glycol, alcohol, anti-microbial components, salicylic acid, alpha acid, antioxidants, including, but not limited to, phenolic compounds, such as chalcones, flavones, flavanones, flavanols, flavonols, dihydroflavonols, isoflavonoids, neoflavonoids, catechins, anthocyanidins, tannins, lignans, aurones, stilbenoids, curcuminoids, alkylphenols, betacyanins, capsacinoids, hydroxybenzoketones, methoxyphenols, naphthoquinones, and phenolic terpenes, resveratrol, curcumin, pinoresinol, ferulic acid, hydroxytyrosol, cinnamic acid, caffeic acid, p-coumaric acid, baicalin (Scutellaria baicalensis root extract), pine bark extract (Pinus pinaster bark/bud extract), ellagic acid, hyaluronic acid and its derivatives, bisabolol, dipotassium glycyrrhizate, glycyrrhizic acid, and vitamins and vitamin derivatives, such as tocopherol and ascorbic acid, and combinations thereof.

In some embodiments, there may be one or more other additives present in the moisturizing peel cream, the other components selected from the group consisting of fillers such as clays, talc, powders, including spherical/silica powders and mineral powders, carbohydrate based gellifying agents, organic thickeners, penetrants, sequestrants, fragrances, dispersants, film-forming agents, ceramides, opacifiers and combinations thereof.

In some embodiments, there may be one or more other components present in the moisturizing peel cream, according to the disclosure, the other components selected from sodium hydroxide, disodium EDTA, sodium citrate, sodium hyaluronate, capryloyl salicylic acid, lactic acid, methyl dihydro jasmonate, acetyl trifluoromethyl phenyl valyglycine, pentaerythrityl tetra-di-t-butyl hydroxydrocinnamate, n-hydroxysuccinimide, palmitoyl oligopeptide, chrysin, palmitoyl tetrapeptide-7, yeast extract, citric acid and combinations thereof.

In some embodiments, additives may include one or a combination of additives selected from the group consisting of sodium hydroxide, trisodium ethylenediamine disuccinate, tocopherol, hydrogen peroxide, and combinations thereof.

In some embodiments, additives may include one or a combination of antimicrobial agents and their salts, selected from, for example, chlorphenesin, caprylyl glycol, phenoxyethanol, caprylhydroxamic acid, benzoic acid, salicylic acid, benzyl alcohol, phenethyl alcohol, benzalkonium chloride, 4-hydroxyacetophenone, piroctone olamine, hexyl glycerin, ethylhexylglycerin, octylglycerin, benzylglycerin, 3-heptoyl-2,2-propandiol, and 1,2-hexandiol, and pentylene glycol, and combinations thereof.

In some particular embodiments, one or more additives in any of the components are selected from the group consisting of sodium hydroxide, trisodium ethylenediamine disuccinate, tocopherol, hydrogen peroxide, chlorophenesin, salicylic acid, phenoxyethanol, bisabolol, dipotassium glycyrrhizate, glycyrrhizic acid, sodium hydroxide, trisodium ethylenediamine disuccinate, tocopherol, sodium hyaluronate, propanediol, hydrogen peroxide, and combinations thereof.

In some embodiments, the moisturizing peel cream is essentially free, or free, or devoid of one or more ingredients selected from the group consisting of silicones, including silicone oils, hydrogen peroxide and its analogs, sulfates, and combinations thereof.

Although the aforementioned optional components are given as an example, it will be appreciated that other optional components compatible with cosmetic applications known in the art may be used.

In accordance with the various embodiments, the number of additives present in the moisturizing peel cream can range from about 0 to about 50%, from about 0.5 to about 30%, from about 1.5 to about 20%, and from about 5 to about 15%, or any suitable combination, sub-combination, range, or sub-range thereof by weight, based on the weight of the moisturizing peel cream or the moisturizing peel cream.

In some embodiments, one or more additives, alone or in combination, can be present in one or both of the moisturizing peel cream and the moisturizing peel cream from about 0.05 to about 50% by weight, from about 0.05% to about 2.5% by weight, from about 0.1 to about 2%, from about 0.25 to about 1.5%, and from about 0.5 to about 1.25%, or any suitable combination, sub-combination, range, or sub-range thereof by weight, based on the weight of the moisturizing peel cream or the moisturizing peel cream.

In some embodiments, one or more other additives, such as preservatives, vitamins, preservatives, and the like, alone or in combination, can be present in one or both of the moisturizing peel cream and the moisturizing peel cream from about 0.05 to about 50% by weight, from about 0.05% to about 25% by weight, from about 0.1 to about 10%, from about 0.25% to about 5%, and from about 0.5 to about 3.5%, or any suitable combination, sub-combination, range, or sub-range thereof by weight, based on the weight of the moisturizing peel cream or the moisturizing peel cream.

Thus, one or a combination of optional additives may be present in the moisturizing peel cream, by weight, based on the weight of the moisturizing peel cream, each one or the combination present from about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.10, 0.20, 0.30, 0.40, 0.50, 0.60, 0.70, 0 0.80, 0.90, 1.0, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 to about 50 weight

percent, including increments and ranges therein and there between.

Methods and Regimens for Providing Skin-Enhancing Benefits; Articles of Manufacture

The moisturizing peel cream may be used according to any one of a number of possible application cycles of a disclosed skin-enhancing regimen wherein the moisturizing peel cream is applied at least once, and more desirable on a daily basis for two or more days and up to 28 days or more. In some embodiments, the moisturizing peel cream may be used in conjunction with other compositions, for example but not limited to an oxidizing component that includes hydrogen peroxide or another oxidizing agent suitable for skin, applied in sequence, once or, or more frequently, with applications of at least one of the components on a daily basis for a few or several days. In some particular embodiments, a skin-enhancing regimen includes application of the moisturizing peel cream at least two or more times, and up to daily applications for a week, or up to three weeks or more. The invention also provides, in various embodiments, a regimen for enhancing skin comprising applying the moisturizing peel cream to skin, including anywhere on the body or on the face. In some embodiments, application on the face may be limited to a region of skin excluding areas of skin around an eye.

In some embodiments, the moisturizing peel cream may be used according to a regimen that includes periodic application of a composition that contains peroxide or another oxidizing agent (an “oxidizing component”). Application of the moisturizing peel cream and oxidizing component may be according to any one of a number of possible application cycles wherein the moisturizing peel cream is used in conjunction with the oxidizing component, applied together, or applied in sequence, and at a frequency of once or, or more frequently, with applications of at least one of the compositions on a daily basis for a few or several days.

In some embodiments, the steps of the regimen further include rinsing the skin after application of at least one of the oxidizing component and the moisturizing peel cream, and include the steps in any order, and wherein the step of rinsing may be repeated.

In some embodiments, the moisturizing peel cream and the oxidizing component are applied sequentially, in any order. In some embodiments, the oxidizing component is applied first, followed by the moisturizing peel cream, and in other embodiments, the moisturizing peel cream is applied first, followed by oxidizing component. In some embodiments, the moisturizing peel cream and the oxidizing component are applied together. In some embodiments, a regimen according to the disclosure may include first application of the oxidizing component on a first day, optionally including application of the moisturizing peel cream on the first day, and thereafter followed by daily applications of the moisturizing peel cream for two or more days.

In some embodiments, the step of applying the oxidizing component is repeated once every third, fourth or fifth day and wherein the step of applying the moisturizing peel cream is skipped on the days that the step of applying the first composition is repeated.

In some embodiments, the regimen is practiced for at least 28 days. The regimen may be selected from one or a combination of application cycles.

Any of the possible application cycles employing the moisturizing peel cream may be achieved using an article of manufacture that includes the moisturizing peel cream, and optionally any other components that may be used with the moisturizing peel cream, packaged in a manner that is suited to the regimen, for example, as a multi-use package, or as one or a plurality of single use packages.

In accordance with the various embodiments, the moisturizing peel cream may be provided in a kit or other article of manufacture.

In various embodiments, provided is an article of manufacture for the moisturizing peel cream comprising at least one contained composition. In some embodiments, the moisturizing peel cream composition is provided in an article of manufacture comprising a container for dispensing the composition, wherein the container is a multi-use or a single use container. In some embodiments, the article of manufacture comprises aliquots of the moisturizing peel cream composition, each separately contained as single units in a plurality of single use containers.

In various embodiments, the moisturizing peel cream composition is contained to be dispensed for application to skin one time, or according to a regimen of daily application

In some embodiments, the moisturizing peel cream is contained as one of a mask, liquid, stick, spray, ointment, paste, foam, mousse, cream, wipe, patch, strip, or film-forming product.

In some embodiments disclosed herein, the moisturizing peel cream may be coupled to an application surface of an applicator. In some embodiments disclosed herein, the application surface may be a surface of a mask configured to conform to a face of a wearer. In some embodiments disclosed herein, the application surface may be selected from the group consisting of a bristle surface of a brush, a roller surface of a rolling applicator, and a blade surface of a spatula.

In some embodiments, the moisturizing peel cream is applied to the keratinous tissue, for example skin of the body or face, in some particular embodiments on the face and generally excluding the eye area, at a temperature of about 15° C. to about 30° C., or from about 20° C. to about 25° C.

The moisturizing peel cream is suitable for home-use or home application. As such, a component according to the present invention is applied by a non-professional or is self-applied in a non-clinical environment.

The moisturizing peel cream may be applied in any manner, for example, by swiping with fingers, pasting, spraying, wiping with a pad, dispensing, etc. (hereinafter “applying”) on the keratinous tissue. This can typically be accomplished with, for example, with fingers, a spray bottle, an absorbent cotton swab wetted with the concentrated solution, with a solution-wetted sable brush or by gentle wiping with a solution-wetted absorbent fibrous material, such as a gauze square or nonwoven pad, but other solution application techniques that coat the skin with the solution, in a uniform manner, are also feasible.

In some embodiments of the moisturizing peel cream which is intended for leave-on application, the composition is applied and allowed to air dry without removal until the user employs their usual cleansing routine. Of course, the moisturizing peel cream may optionally be removed after application and some selected interval of time, though the results may not be as desirable. Drying may be promoted by directing a gentle stream of air, warm air, onto the treated area or by other analogous procedures. A single uniform application of the moisturizing peel cream to the keratinous tissue is generally sufficient. Additional or multiple applications either before or immediately after the applied solution has dried are normally unnecessary but may be useful in some situations, e.g., in applications to keratinous tissue on other parts of the body other than the face or in applications to keratinous tissue severely in need of peeling. For rinse-off forms of the moisturizing peel cream, the composition should be rinse off, in particular prior to applying any other composition or product to the skin.

### EXAMPLES

### Example 1: Raw Materials

Percentages of each ingredient as may be exemplified in the following examples are shown as amount of active, wherein the raw materials may be present in an amount that is equal to the amount of active, or if the raw material has a concentration of active that is less than 100% then the composition includes the raw material that includes active and a suitable solvent, wherein the concentration of active in the raw material is provided herein below.

The following raw materials for ingredients as shown in the TABLES herein include actives present at less than 100%, as follows:

TABLE 1
Representative Raw Materials Percent Active

| | Percent Active |
|---|---|
| Raw Material | (%) |
| Stearic Acid | 53 |
| Trisodium Ethylenediamine Disuccinate | 37 |
| Hydroxyethyl Acrylate/Sodium Acryloyldimethyl Taurate | 90 |
| Copolymer | |
| Glycolic Acid | 70 |

### Example 2: Compositions

Moisturizing peel cream: Emulsion Cream and Liquid Forms

The various embodiments of the inventive composition as exemplified in Table 2 include the foregoing raw materials wherein the active constituents of the raw materials are listed as discrete ingredients, wherein the amounts of active ingredients are given for the exemplified embodiments as % by weight of the active ingredients, not as amounts of raw materials except as may be expressly stated.

TABLE 2
Inventive Embodiments

| | | 888006 32YP | 888006 34CL | | | |
|---|---|---|---|---|---|---|
| | | Peel Cream | Peel Cream | 888006 5G47 | | |
| | 888006 32Y | based on | based on | Peel moisturizer + | 888006 5A | 888006 5G |
| | Peel moisturizer | 32YZ w/o | 34CK w/o | non-polar oil + cocoa | AHA/BHA | Peel moisturizer |
| | 6% GA + 4% GDL | bisabolol | bisabolol | butter + soothing | Moisturizer | with 7% glycerin |
| INCI US | INV1 | INV2 | INV3 | INV4 | INV5 | INV6 |
| TOCOPHEROL | 0.50 | 0.50 | 0.50 | 0.50 | 0.25 | 0.50 |
| CHLORPHENESIN | | | | | 0.20 | 0.20 |
| HYDROXYETHYL ACRYLATE/ | 3.56 | 3.56 | 3.56 | 3.56 | 3.56 | 3.56 |
| SODIUM ACRYLOYLDIMETHYL | | | | | | |
| TAURATE COPOLYMER | | | | | | |
| XANTHAN GUM | 0.20 | 0.20 | 0.20 | 0.20 | 0.20 | 0.20 |
| PHENOXYETHANOL | 0.50 | 0.50 | 0.30 | 0.50 | 0.60 | 0.60 |
| SODIUM HYDROXIDE | 2.20 | 2.20 | 2.20 | 2.65 | 2.25 | 2.25 |
| TRISODIUM ETHYLENEDIAMINE | 0.037 | 0.037 | 0.037 | 0.037 | 0.037 | 0.037 |
| DISUCCINATE | | | | | | |
| BISABOLOL | 0.50 | | | 0.50 | | |
| METHYLDIHYDROJASMONATE | | 0.50 | 0.50 | | | |
| GLYCERYL STEARATE | 2.00 | 2.00 | 1.50 | 2.00 | 2.00 | 2.00 |
| THEOBROMA GRANDIFLORUM | | 3 | 3 | | | |
| SEED BUTTER | | | | | | |
| MYRISTIC ACID | 0.03 | 0.03 | 0.03 | 0.03 | 0.03 | 0.03 |
| GLYCERIN | 7.00 | 7.00 | 7.00 | 7.00 | 10.00 | 7.00 |
| PALMITIC ACID | 0.44 | 0.44 | 0.44 | 0.44 | 0.44 | 0.44 |
| STEARIC ACID | 0.53 | 0.53 | 0.53 | 0.53 | 0.53 | 0.53 |
| TOCOPHEROL | | 0.0015 | 0.0015 | | | |
| CETEARYL ALCOHOL | 0.71 | 0.71 | 0.71 | 0.71 | 0.71 | 0.71 |
| C12-15 ALKYL BENZOATE | | | | | 4.00 | 4.00 |
| CETEARETH-20 | 0.29 | 0.29 | 0.29 | 0.29 | 0.29 | 0.29 |
| BUTYROSPERMUM PARKII | 2 | 2 | 2 | 2 | 2 | 2 |
| (SHEA) BUTTER | | | | | | |
| SALICYLIC ACID | 0.45 | 0.45 | 0.45 | 0.45 | 0.45 | 0.45 |
| SORBITAN ISOSTEARATE | 0.22 | 0.22 | 0.22 | 0.22 | 0.22 | 0.22 |
| CAPRYLIC/CAPRIC | | | | | 5.00 | 5.00 |
| TRIGLYCERIDE | | | | | | |
| WATER | 55.11 | 55.11 | 62.81 | 56.67 | 49.02 | 51.77 |
| CITRIC ACID | 0.00002 | 0.00002 | 0.00002 | 0.00002 | 0.00002 | 0.00002 |
| GLYCOLIC ACID | 6 | 6 | 6 | 10 | 10 | 10 |
| HELIANTHUS ANNUUS | 5.00 | | | 2.00 | | |
| (SUNFLOWER) SEED OIL | | | | | | |
| THEOBROMA CACAO | 3.00 | | | 3.00 | 1.00 | 1.00 |
| (COCOA) SEED BUTTER | | | | | | |
| GLYCERYL STEARATE | 1.00 | 1.00 | 0.50 | 1.00 | 1.00 | 1.00 |
| PEG-100 STEARATE | 1.00 | 1.00 | 0.50 | 1.00 | 1.00 | 1.00 |
| POLYSORBATE 20 | 0.50 | 0.50 | 0.50 | 0.50 | 2.00 | 2.00 |
| POLYSORBATE 60 | 0.22 | 0.22 | 0.22 | 0.22 | 0.22 | 0.22 |
| C15-19 ALKANE | | | 1.00 | 4.00 | | |
| GLUCONOLACTONE | 4.00 | 4.00 | 4.00 | | | |
| ALUMINUM STARCH | 2.00 | 2.00 | 1.00 | | | |
| OCTENYLSUCCINATE | | | | | | |
| COCO-CAPRYLATE/CAPRATE | 1.00 | 6.00 | 1.00 | | 3.00 | 3.00 |

### Additional Inventive Embodiments

The various embodiments of the inventive composition as exemplified in Tables 3 and 4 include the above listed raw materials wherein the active constituents of the raw materials are listed as amounts of raw materials present by weight of the entire composition, and not the final amounts of active ingredients.

TABLE 3
Additional Inventive Embodiments

| INGREDIENT | INV7 | INV8 | INV9 | INV10 | INV11 |
|---|---|---|---|---|---|
| STEARIC ACID | 1.00 | 1.00 | 1.00 | 14.28 | 14.28 |
| POLYSORBATE 20 | 0.50 | 0.50 | 1.00 | 0.45 | 0.45 |
| GLYCERYL STEARATE (and) | 2.00 | 2.00 | 2.00 | 10.00 | 7.00 |
| PEG-100 STEARATE | | | | | |
| GLYCERYL STEARATE | 2.00 | 2.00 | 2.00 | 2.00 | 2.22 |
| CETEARYL ALCOHOL (and) | 1.00 | 1.00 | 1.00 | 2.00 | 2.00 |
| CETEARETH-20 | | | | | |
| GLYCOLIC ACID | 8.57 | 8.57 | 8.57 | 2.00 | 1.67 |
| GLUCONOLACTONE | 4.00 | 4.00 | 4.00 | 1.00 | 1.11 |
| SALICYLIC ACID | 0.45 | 0.45 | 0.45 | 1.00 | 1.00 |
| GLYCERIN | 7.00 | 7.00 | 7.00 | 5.00 | 0 |
| COCO-CAPRYLATE/ | 1.00 | 1.00 | 1.00 | 3.00 | 3.00 |
| CAPRATE | | | | | |
| ALUMINUM STARCH | 2.00 | 2.00 | 2.00 | 4.00 | 4.00 |
| OCTENYLSUCCINATE | | | | | |
| XANTHAN GUM | 0.20 | 0.20 | 0.20 | 2.00 | 0.50 |
| HYDROXYETHYL ACRY- | 4.00 | 4.00 | 4.00 | 1.00 | 1.00 |
| LATE/SODIUM ACRYLOYL- | | | | | |
| DIMETHYL TAURATE CO- | | | | | |
| POLYMER | | | | | |
| HELIANTHUS ANNUUS | 5.00 | 5.00 | 5.00 | | |
| SEED OIL | | | | | |
| BUTYROSPERMUM PARKII | 2.00 | 2.00 | 2.00 | 0.20 | 0.20 |
| BUTTER | | | | | |
| THEOBROMA CACAO SEED | 3.00 | 3.00 | 3.00 | 4.00 | 4.00 |
| BUTTER | | | | | |
| PHENOXYETHANOL | 0.50 | 0.50 | 0.50 | 2.25 | 2.25 |
| SODIUM HYDROXIDE | 2.20 | 2.20 | 2.20 | 0.25 | 0.50 |
| TOCOPHEROL | 0.50 | 0.50 | 0.50 | 0.20 | 0.20 |
| TRISODIUM ETHYLENEDI- | 0.10 | 0.10 | 0.10 | 0.60 | 0.60 |
| AMINE DISUCCINATE | | | | | |
| PENTYLENE GLYCOL | | 2.50 | | 0.10 | 0.10 |
| BISABOLOL | 0.50 | 0.50 | 0.50 | 44.67 | 53.92 |
| DODECANE | | 1.00 | | | |
| WATER | 52.48 | 48.98 | 51.98 | | |

TABLE 4
Additional Inventive Embodiments

| Ingredient | INV12 | INV13 | INV14 |
|---|---|---|---|
| GLYCOLIC ACID | 14.28 | 14.28 | 14.28 |
| GLYCERYL STEARATE | 0.45 | 2.00 | 2.00 |
| STEARIC ACID | 7.00 | 1.00 | 1.00 |
| HELIANTHUS ANNUUS SEED OIL | 1.62 | 2.00 | |
| POLYSORBATE 20 | 2.00 | 0.50 | 2.00 |
| GLYCERYL STEARATE (and) PEG- | 1.43 | 2.00 | 2.00 |
| 100 STEARATE | | | |
| SALICYLIC ACID | 1.25 | 0.45 | 0.45 |
| CAPRYLIC/CAPRIC TRIGLYCERIDE | 1.00 | | 5.00 |
| C15-19 ALKANE | 0 | 4.00 | |
| COCO-CAPRYLATE/CAPRATE | 6.00 | | 3.00 |
| C12-15 ALKYL BENZOATE | 0 | | 4.00 |
| BUTYROSPERMUM PARKII BUTTER | 2.00 | 2.00 | 2.00 |
| GLYCERIN | 1.00 | 7.00 | 7.00 |
| THEOBROMA CACAO SEED BUTTER | 2.00 | 3.00 | 1.00 |
| CETEARYL ALCOHOL (and) | 0.20 | 1.00 | 1.00 |
| CETEARETH-20 | | | |
| XANTHAN GUM | 4.00 | 0.20 | 0.20 |
| HYDROXYETHYL ACRYLATE/ | 2.25 | 4.00 | 4.00 |
| SODIUM ACRYLOYLDIMETHYL | | | |
| TAURATE COPOLYMER | | | |
| TRISODIUM ETHYLENEDIAMINE | 0.50 | 0.10 | 0.10 |
| DISUCCINATE | | | |
| TOCOPHEROL | 0.20 | 0.50 | 0.50 |
| SODIUM HYDROXIDE | 0.60 | 2.65 | 2.25 |
| CHLORPHENESIN | 0.10 | | 0.20 |
| PHENOXYETHANOL | 52.12 | 0.50 | 0.60 |
| BISABOLOL | | 0.50 | |
| WATER | | 52.32 | 47.42 |

The composition as exemplified in Table 5 include raw materials wherein the active constituents of the raw materials are listed as discrete ingredients, wherein the amounts of active ingredients are given as % by weight of the active ingredients, not as amounts of raw materials.

TABLE 5
Test Composition Lacking Lamellar
Structure in Emulsion (See FIG. 2)

| 888006 5G16A | |
|---|---|
| INGREDIENT | Amount |
| WATER | 59.42 |
| GLYCERIN | 7.00 |
| SODIUM HYDROXIDE | 2.25 |
| SALICYLIC ACID | 0.45 |
| GLYCOLIC ACID | 14.28 |
| HYDROXYETHYL ACRYLATE/ | 4.00 |
| SODIUM ACRYLOYLDIMETHYL | |
| TAURATE COPOLYMER | |
| XANTHAN GUM | 0.20 |
| C12-15 ALKYL BENZOATE | 4.00 |
| COCO-CAPRYLATE/CAPRATE | 3.00 |
| THEOBROMA COCAO (COCOA) | 1.00 |
| SEED BUTTER | |
| STEARIC ACID | 1.00 |
| GLYCERYL STEARATE (AND) | 2.00 |
| PEG-100 STEARATE | |
| CETEARYL ALCOHOL (AND) | 0.50 |
| CETEARETH-20 | |
| TRISODIUM ETHYLENEDIAMINE | 0.10 |
| DISUCCINATE | |
| PHENOXYETHANOL | 0.60 |
| CHLORPHENESIN | 0.20 |

### Example 3: In Vitro Stratum Corneum Testing for Lipid Disorganization w/Cream and Toner Forms of Moisturizing Peel Cream

In Vitro Stratum Corneum (SC) testing to evaluate lipid disorganization was conducted, and AT was determined using differential scanning calorimetry (DSC), measuring the heat change associated with thermal denaturation when heated at a constant rate. Inventive moisturizing peel cream samples and control samples were applied on in vitro SC surface (˜0.1 g/sample) for 15 min at 75% relative humidity. Following single or sequential applications, the SC surface was wiped clean and rinsed with distilled water and allowed to dry overnight at 75% relative humidity. The DSC profile was measured from 20° C. to 120° C. at 5° C/min to provide AT for lipid related peaks at 70-75° C. and 80-85° C. AT is calculated by the change in peak temperature by comparing the temperature of untreated control versus samples treated with test compositions.

The results show that after one application of an embodiment of the inventive moisturizing peel cream composition, exfoliation and improved skin brightness were demonstrated to be comparable to a 30% acid professional peel.

### Example 4: Clinical Study

Compositions that include inventive compositions INV 1 and INV4 and suitable control compositions were evaluated in an in vivo clinical study to show biological effects on live skin.

In the study, an application regimen included a once daily application of an embodiment of the moisturizing peel cream in leave on moisturizer form.

TABLE 6
Clinical Result of INV1 compared to 30% glycolic acid
professional peel, each vs Baseline (no treatment)

| Clinical Grading | INV1 vs. Baseline | | | | 30% GA Pro Peel vs. Baseline | | | |
|---|---|---|---|---|---|---|---|---|
| Assessment | Day 3 | Week 1 | Week 2 | Week 4 | Day 3 | Week 1 | Week 2 | Week 4 |
| Dark Spot Intensity | | X | X | Y | X | X | X | Y |
| PIH | | | | | | | | |
| Overall | | X | Y | Y | | | | Y |
| Hyperpigmentation | | | | | | | | |
| Skin Tone Evenness | X | Y | Y | Y | X | Y | Y | Y |
| Radiance | Y | Y | Y | Y | Y | Y | Y | Y |
| Global Fine Lines | X | Y | Y | Y | X | Y | Y | Y |
| Global Wrinkles | X | X | X | Y | | X | X | Y |
| Overall Appearance | Y | Y | Y | Y | Y | Y | Y | Y |
| of Skin | | | | | | | | |
| Appearance of Pores | X | Y | Y | Y | Y | Y | Y | Y |

X indicates statistically significant improvement when compared to baseline (p ≤ 0.05);
Y indicates statistical and clinical significance (minimum change of −0.51) and (p ≤ 0.05).

As shown in the foregoing Table 6, statistically significant improvement as compared with baseline at virtually every time point for each of the graded parameters, and clinically significant improvement in all graded parameters at week 4 and in the case of radiance and overall appearance as soon as day 3. These results are surprisingly comparable to the professional 30% glycolic acid peel and without the attendant expense and associated harshness of the professional peel.

The articles “a” and “an,” as used herein, mean one or more when applied to any feature in embodiments of the present invention described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used. The adjective “any” means one, some, or all indiscriminately of whatever quantity.

“At least one,” as used herein, means one or more and thus includes individual components as well as mixtures/combinations.

The transitional terms comprising,” “consisting essentially of” and “consisting of,” when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed invention. All materials and methods described herein that embody the present invention can, in alternate embodiments, be more specifically defined by any of the transitional terms “comprising,” “consisting essentially of,” and “consisting of.”

Other than in the operating examples, or where otherwise indicated, all numbers expressing quantities of ingredients and/or reaction conditions are to be understood as being modified in all instances by the term “about,” meaning within 10% of the indicated number (e.g. “about 10%” means 9%-11% and “about 2%” means 1.8%-2.2%).

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used, and may or may not reflect the final percentage of the active, wherein the final percentage of the active is dependent on the weight percent of active in the raw material.

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. Further, it is understood that when an amount of a component is given, it is intended to signify the amount of the active material unless otherwise specifically stated.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements. The example that follows serves to illustrate embodiments of the present disclosure without, however, being limiting in nature.

While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. A moisturizing peel cream composition comprising:
at least one hydroxy acid, comprising at least one alpha-hydroxy acid, present in an amount that is at least 5.5% by weight, based on the weight of the composition;
at least one lightweight emollient selected from the group consisting of polar emollients, liquid alkanes, and combinations thereof;
a blend of surfactants, the blend of surfactants comprising one or more surfactants selected from the group consisting of cetearyl alcohol, ceteareth-20, glyceryl stearate and PEG-100 stearate, and combinations thereof;
at least one thickening polymer, the total amount of thickening polymer present in an amount that is at least 3% by weight, based on the weight of the composition; and
at least one water based solvent, the at least one water based solvent comprising water present from about 40% to about 75% by weight, based on the weight of the composition,
wherein the moisturizing peel cream composition is an oil in water emulsion comprising a lamellar liquid crystal structure and has a pH of about 3.5 to about 4.1.

2. The composition according to claim 1, wherein the composition is provided for leave-on application to skin according to a daily treatment regimen of at least one day to up to twenty eight (28) days or more.

3. The composition according to claim 1, wherein the composition is provided in a creamy leave on form that is free or essentially free from silicones.

4. The composition according to claim 1, wherein the at least one water based solvent comprises water, present in the composition in a range from about 40% to about 75% by weight, based on the total weight of the composition.

5. The composition according to claim 1, wherein the at least one water based solvent comprises glycerin, present in the composition in a range from about 1% to about 20% by weight, based on the total weight of the composition.

6. The composition according to claim 1, further comprising at least one additional hydroxy acid selected from the group consisting of alph-, poly-, and beta-hydroxy acids, and combinations thereof.

7. The composition according to claim 1, wherein the at least one alpha-hydroxy acid comprises glycolic acid, present from at least about 5.5% to about 15% by weight, based upon the total weight of the composition.

8. The composition according to claim 1, further comprising at least one hydroxy acid selected from the group consisting of poly-hydroxy acid comprising gluconolactone, present from about 1% to about 15% by weight, based upon the total weight of the composition, and beta-hydroxy acid comprising salicylic acid, present from about 0.1% to about 2% by weight, based upon the total weight of the moisturizing peel cream.

9. The composition according to claim 1, wherein the blend of surfactants is present in a range from about 4.5% to about 8%.

10. The composition according to claim 1, wherein the at least one light-weight emollient is present in a range from about 1% to about 12% and comprises coco-caprylate/caprate.

11. The composition according to claim 1, wherein the at least one or more polymers comprises xanthan gum, and Hydroxyethyl Acrylate/Sodium Acryloyldimethyl Taurate Copolymer, sclerotium gum, hydroxypropyl starch phosphate, sodium carboxymethyl starch.

12. The composition according to claim 1, further comprising from about 5% to about 20% of one or a combination of emollients in addition to the at least one light-weight emollient.

13. The composition according to claim 14, wherein each of the of emollients in addition to the at least one light-weight emollient is selected from the group consisting of plant butters, oils, and combinations thereof.

14. The composition according to claim 13, wherein each of the of emollients in addition to the at least one light-weight emollient is selected from the group consisting of Helianthus annuus seed oil, Butyrospermum parkii butter, Theobroma grandiflorum seed butter, Theobroma cacao seed butter, dodecane, caprylic/capric triglyceride, C12-15 alkyl benzoate, sunflower seed oil and combinations thereof.

15. The composition according to claim 1, wherein the composition is provided in a creamy leave on form that is free or essentially free from silicones, and wherein the at least one water based solvent comprises water, present in the composition in a range from about 40% to about 75% by weight, based on the total weight of the composition and glycerin, present in the composition in a range from about 1% to about 20% by weight, based on the total weight of the composition an wherein the at least one alpha-hydroxy acid comprises glycolic acid, present from at least about 5.5% to about 15% by weight, based upon the total weight of the composition, the composition further comprising at least one hydroxy acid selected from the group consisting of poly-hydroxy acid comprising gluconolactone, present from about 1% to about 15% by weight, based upon the total weight of the composition, and beta-hydroxy acid comprising salicylic acid, present from about 0.1% to about 2% by weight, based upon the total weight of the composition, and wherein the blend of surfactants is present in a range from about 4.5% to about 8%, and wherein the at least one light-weight emollient is present in a range from about 1% to about 12% and comprises coco-caprylate/caprate, and wherein the at least one or more polymers comprises aluminum starch octenylsuccinate, xanthan gum, and Hydroxyethyl Acrylate/Sodium Acryloyldimethyl Taurate Copolymer, the composition further comprising from about 5% to about 20% of one or a combination of emollients in addition to the at least one light-weight emollient selected from the group consisting of plant butters, oils, and combinations thereof, and wherein the composition optionally comprises one or more additional ingredients selected from the group consisting of chelating agents, pH adjusters, humectants, antioxidants, plant extracts, plant oils and butters, active components, essential oils, vitamins, antimicrobials and preservatives, fragrances, pearlescent agents, odor absorbers, coloring materials, fillers such as clays, talc, powders, including spherical/silica powders and mineral powders, carbohydrate based gellifying agents, organic thickeners, penetrants, sequestrants, fragrances, dispersants, film-forming agents, ceramides, opacifiers, and combinations thereof.

16. An article of manufacture for the skin-enhancing system comprising: at least a first contained composition comprising the moisturizing peel cream according to claim 1.

17. An article of manufacture according to claim 16, wherein the moisturizing peel cream composition is contained to be dispensed for application to skin in a container selected from the group consisting of a multi-use container, a single use container and a plurality of single use containers.

18. A regimen for enhancing skin, comprising: applying the moisturizing peel cream according to claim 1 to skin, including anywhere on a body or on a face or on a face and limited to a region of skin excluding areas of skin around an eye.

19. The regimen for enhancing skin according to claim 18, the regimen including application of the composition on a daily basis the regimen further including periodic application of a composition that contains an oxidizing component, wherein the moisturizing peel cream and oxidizing component may be applied together, or applied in sequence, on a daily basis for a few or several days.

20. The regimen for enhancing skin according to claim 19, the regimen further including rinsing the skin after application of at least one of the oxidizing component and the moisturizing peel cream.
